(19) United States
(12) Patent Application Publication
(10) Pub. No.: US 2026/0183458 A1
(43) Pub. Date: Jul. 2, 2026

(54) Breast Pump

(71) Applicant: Shenzhen Oceanwing Smart Innovations Technology Co., Ltd., Shenzhen (CN)

(72) Inventors: Shanshan Xiao, Shaoguan (CN); Han Lin, Shenzhen (CN); Yingying Qiao, Shaoguan (CN); Zhihai Zhang, Beijing (CN); Liying Chen, Shaoguan (CN)

(21) Appl. No.: 19/552,191

(22) Filed: Feb. 27, 2026

Related U.S. Application Data
(63) Continuation of application No. 18/413,559, filed on Jan. 16, 2024.

(30) Foreign Application Priority Data
Jan. 16, 2023 (CN) 202310102123.5
Jan. 16, 2023 (CN) 202320160064.2
Jan. 16, 2023 (CN) 202320192428.5

(51) Int. Cl. A61M 1/06

(52) U.S. Cl. CPC A61M 1/064; A61M 2205/36

(57) ABSTRACT

The present disclosure relates to a breast pump comprising a suction mechanism and a main body. The suction mechanism comprises an elastic bowl and a suction assembly, the elastic bowl having an accommodation cavity and being at least partially accommodated in the suction assembly. The main body comprises a boss sealingly fitting with the accommodation cavity of the elastic bowl, the boss being provided with a suction hole that is in communication with the accommodation cavity to create a connected passageway. When the main body extracts air through the suction hole and the accommodation cavity decreases so that the elastic bowl contracts, there is no gap between the elastic bowl and the boss, and negative pressure is generated in the suction assembly.

### CROSS-REFERENCE TO RELATED APPLICATION

The present application is a continuation of U.S. application Ser. No. 18/413,559, filed Jan. 16, 2024, which claims priority of CN Application Nos. 202310102123.5, 202320192428.5 and 202320160064.2, all filed on Jan. 16, 2023. The above applications are incorporated by reference in their entirety.

### FIELD

This application relates to the field of breast pump technology, in particular to a breast pump.

### BACKGROUND

Breast pumps can be used to suck milk from pregnant women. The working principle of the breast pump is generating negative pressure mainly by a main body to suck milk. However, with regard to traditional breast pumps, during the process of generating negative pressure, part of energy of the main body does not contribute to the generation of negative pressure, so the main body will do certain idle work, thereby affecting an energy utilization rate of the breast pump.

### SUMMARY

This disclosure provides a breast pump comprising: a suction mechanism, comprising a suction assembly and an elastic bowl which is at least partially accommodated in the suction assembly; and a main body, comprising a boss sealingly fitting with an accommodation cavity of the elastic bowl, the boss being provided with a suction hole that is in communication with the accommodation cavity to create a connected passageway, wherein when the main body extracts air through the suction hole and the accommodation cavity decreases so that the elastic bowl contracts, there is no gap between the elastic bowl and the boss, and negative pressure is generated in the suction assembly.

In some examples, the elastic bowl comprises a main bowl and a flange, the main bowl is accommodated in the suction assembly and forms the accommodation cavity, the flange is annular and is curved and connected with the main bowl, and there is an annular gap between the main bowl and the flange that fits with the suction assembly.

In some examples, the boss has a limiting surface disposed toward a bottom of the elastic bowl, the elastic bowl after contraction can be attached to the limiting surface, and the suction hole has an extraction opening on the limiting surface which is connected with the accommodation cavity.

In some examples, the suction assembly comprises a storage bin, a suction cover and a control valve. The main body, the elastic bowl, the suction cover and the control valve are all disposed on the storage bin; an adjustment cavity is provided between the elastic bowl and the storage bin, and a storage cavity is provided between the suction cover and the storage bin. When the elastic bowl contracts, the control valve is closed, and the adjustment cavity generates negative pressure and is isolated from the storage cavity; and when the elastic bowl expands, the control valve is opened, the negative pressure of the adjustment cavity disappears, and the adjustment cavity is in fluid communication with the storage cavity.

In some examples, the storage bin comprises a flow nozzle. When the breast pump is in a working state, an angle between an axial direction of the flow nozzle and a direction of gravity is 80° to 90°, and the control valve is sleeved on the flow nozzle.

In some examples, the control valve comprises a fixed cylinder sleeved on the flow nozzle. The fixed cylinder has an end surface and a stop surface spaced apart along the axial direction. The stop surface is located in the fixed cylinder, the end surface is closer to the elastic bowl than the stop surface, the stop surface abuts an end of the flow nozzle, and the end surface comprises a first section and a second section arranged at intervals. The storage bin has a mounting surface, and the flow nozzle is protrudingly disposed on the mounting surface. The first section is farther from the stop surface than the mounting surface, and a distance from the mounting surface to the stop surface is greater than or equal to a distance from the second section to the stop surface.

In some examples, the control valve further comprises an opening and closing cylinder connected with the fixed cylinder. The opening and closing cylinder each has a first inner surface and a second inner surface spaced apart. The first inner surface is disposed close to the first section, the second inner surface is disposed close to the second section and forms an angle with an axis of the fixed cylinder. A distance between the first inner surface and the second inner surface decreases along a direction where the fixed cylinder points to the opening and closing cylinder; when the first inner surface and the second inner surface fit, the control valve is closed; when the first inner surface and the second inner surface separate, the control valve is opened.

In some examples, the suction cover comprises a hard cover and a soft cover, and the soft cover is more flexible than the hard cover. The soft cover is inserted into the storage bin, and the hard cover is disposed on an edge of the soft cover and is snap-connected with the storage bin.

In some examples, the soft cover comprises a fitting part and a mounting part which is protrudingly disposed on the fitting part, inserted into the storage bin, and provided with a guide hole that is in fluid communication with the adjustment cavity, and the fitting part abuts the storage cavity to seal the storage cavity.

In some examples, at least one of the following solutions is also included: the storage bin is integrally formed; and the suction assembly further comprises a plug, the suction cover is provided with a liquid outlet hole that is in fluid communication with the storage cavity, and the plug fits with the liquid outlet hole.

In some examples, the breast pump further comprises: a heating element disposed on the suction cover and electrically connected with the main body.

In some examples, the heating element is embedded in the suction cover.

In some examples, the heating element comprises a heating part and a conductive part connected with each other, and the conductive part is electrically connected with the main body.

In some examples, the conductive part has a columnar structure. When the breast pump is in a working state, the conductive part extends in the direction of gravity; or the conductive part extends in a direction perpendicular to the direction of gravity.

In some examples, the suction cover comprises a protruding column, the conductive part penetrates the protruding column, the protruding column is inserted into the storage bin, and the conductive part is away from an end of the heating part and abuts the main body.

In some examples, the heating part is a spiral resistance wire.

In some examples, the heating element further comprises a heat conducting sheet, the heating part is a flexible heating sheet, and the heat conducting sheet adheres to the flexible heating sheet.

In some examples, the heating element comprises a heating part attached to an outer surface of the suction cover.

In some examples, the heating element further comprises a fixed base and a conductive part. The fixed base is detachably connected with the main body. The conductive part is connected with the heating part, penetrates the fixed base, has a columnar structure, and is electrically connected with the main body.

In some examples, at least one of the following solutions is also included: the heating element is magnetically connected with the main body; the breast pump further comprises an adjustment plug which is inserted into the suction cover and covers the heating part, the adjustment plug is provided with an annular step surface, and the heating part abuts the step surface; and the heating element is detachably connected with the main body and the suction cover, and the heating part is attached to an outer surface of the suction cover which is in contact with the human body.

In some examples, the breast pump further comprises a temperature sensor disposed on the heating element and electrically connected with the main body.

In some examples, the breast pump further comprises: a heating element disposed on at least one of the storage bin and the main body, wherein the heating element is electrically connected with the main body.

In some examples, when the heating element is disposed on the storage bin, the storage bin comprises a protrusion protruding toward the suction cover, the heating part is accommodated in the protrusion, and the protrusion abuts the suction cover.

In some examples, the heating element further comprises a conductive part connected with the heating part, the storage bin has a bearing surface for bearing the main body, the bearing surface is provided with a sink hole, at least part of the conductive part is accommodated in the sink hole, and the conductive part is electrically connected with the main body.

In some examples, when the breast pump is in a working state, the bearing surface is perpendicular to the direction of gravity.

In some examples, a surface of the conductive part is aligned with the bearing surface.

In some examples, the conductive part is provided with a contact electrically connected with the main body.

In some examples, when the heating element is disposed on the main body, the main body comprises a first bulging part bulging toward the storage bin, which comprises a second bulging part bulging toward the suction cover. The heating part is accommodated in the first bulging part, which is accommodated in the second bulging part, and the second bulging part abuts the suction cover.

In some examples, the heating element is embedded in the storage bin or the main body by in-mold injection molding.

A technical effect of an example of the present disclosure is: when the accommodation cavity decreases so that the elastic bowl contracts, a gap between the elastic bowl and the boss is approximately zero. Thus, almost all energy of air extraction by the main body is converted into negative pressure, substantially eliminating the air pump from extracting air in the gap between the main bowl and the boss that cannot contribute to the negative pressure, thereby substantially eliminating the idle work of the air pump and improving the utilization rate of energy by the air pump and the entire breast pump. As the energy utilization rate is improved, when output power of the main body is the same, the breast pump 10 can suck more milk per unit time.

### BRIEF DESCRIPTION OF THE DRAWINGS

FIG. 1 is a perspective view of a breast pump provided in a first example;

FIG. 2 is a perspective section view of the breast pump shown in FIG. 1;

FIG. 3 is a plan section view of the breast pump shown in FIG. 1;

FIG. 4 is an exploded view of a first example of the breast pump shown in FIG. 1;

FIG. 5 is a perspective section view of FIG. 4;

FIG. 6 is an exploded view of a second example of the breast pump shown in FIG. 1;

FIG. 7 is a perspective view of a control valve in the breast pump shown in FIG. 1;

FIG. 8 is a perspective section view of the control valve in the breast pump shown in FIG. 1;

FIG. 9 is a plan section view of the control valve in the breast pump shown in FIG. 1;

FIG. 10 is a perspective view of a main body of the breast pump shown in FIG. 1;

FIG. 11 is a perspective view of a breast pump provided in a second example;

FIG. 12 is an exploded view of the breast pump shown in FIG. 11;

FIG. 13 is an exploded view of the breast pump shown in FIG. 11 from a other viewing angle;

FIG. 14 is a plan section view of the breast pump shown in FIG. 11;

FIG. 15 is a perspective view of a breast pump provided in a third example;

FIG. 16 is an exploded view of the breast pump shown in FIG. 11 with the main body removed;

FIG. 17 is a perspective view of a breast pump provided in a fourth example;

FIG. 18 is an exploded view of the breast pump shown in FIG. 17;

FIG. 19 is a perspective section view of the breast pump shown in FIG. 17;

FIG. 20 is a plan section view of the breast pump shown in FIG. 17 with the main body and storage bin removed;

FIG. 21 is an exploded view of a breast pump provided in a fifth example;

FIG. 22 is an exploded view of the breast pump in FIG. 21 from the other viewing angle;

FIG. 23 is an exploded view of a breast pump provided in a sixth example; and

FIG. 24 is an exploded view of the breast pump in FIG. 23 from the other viewing angle.

### DETAILED DESCRIPTION

In order to facilitate understanding of the present disclosure, the present disclosure will be described more fully below with reference to the relevant drawings. The examples of the present disclosure are shown in the accompanying drawings. However, the present disclosure may be implemented in many different forms and is not limited to the examples described herein. Rather, these examples are provided to provide a thorough and comprehensive understanding of the disclosure of the present disclosure.

It should be noted that when an element is referred to as being “fixed” to another element, it can be directly on the other element or intervening elements may also be present. When an element is said to be “connected” with another element, it can be directly connected with the other element or there may also be intervening elements present. The terms “inner”, “outer”, “left”, “right” and similar expressions used herein are for illustrative purposes only and do not represent the only implementation manner.

### FIRST EXAMPLE

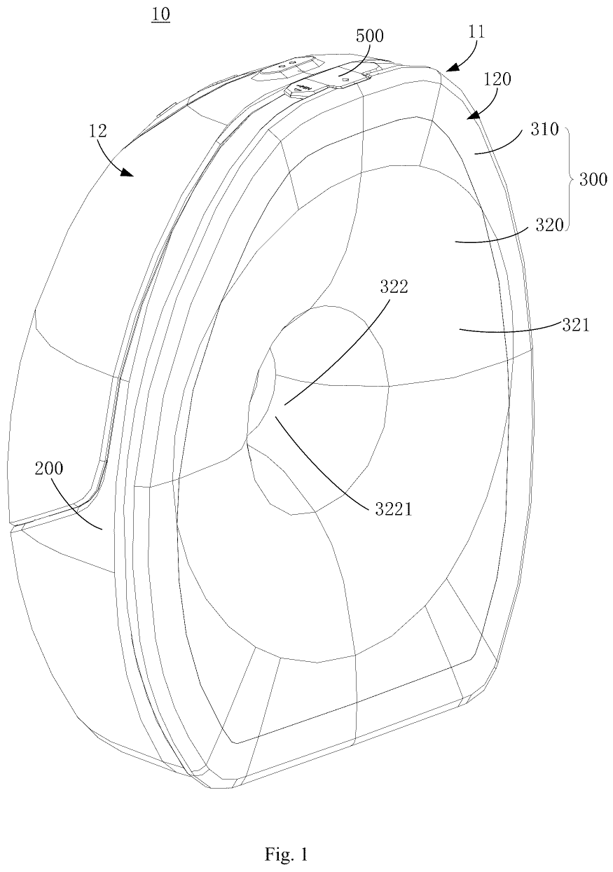

Referring to FIGS. 1, 2 and 3, the breast pump 10 provided in the first example of the disclosure may be a wearable device, that is, the breast pump 10 can be directly worn on a human's breast to suck milk. The breast pump 10 comprises a suction mechanism 11 worn on the breast and a main body 12 disposed on the suction mechanism 11. The suction mechanism 11 comprises an elastic bowl 110 and a suction assembly 120 which comprises a storage bin 200, a suction cover 300, a control valve 400 and a plug 500. The main body 12, the elastic bowl 110, the suction cover 300, the control valve 400 and the plug 500 can all be disposed on the storage bin 200 by detachable connecting.

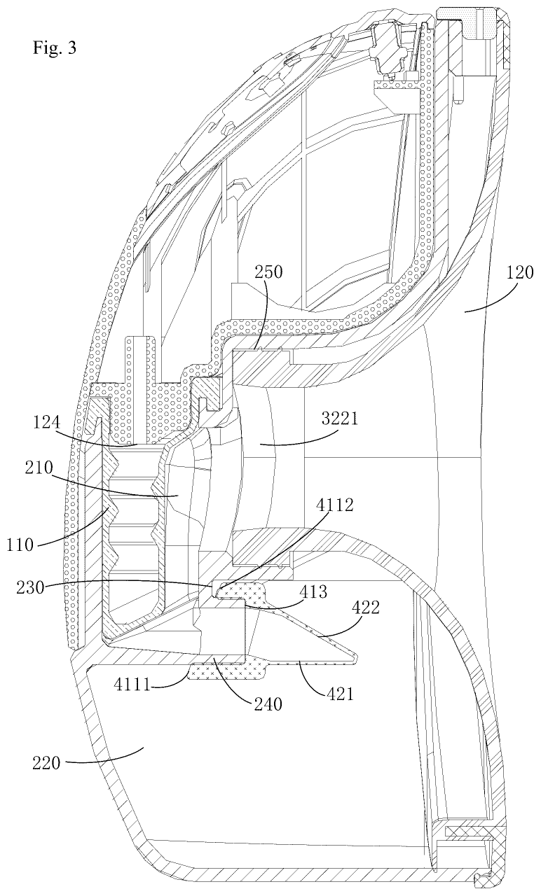

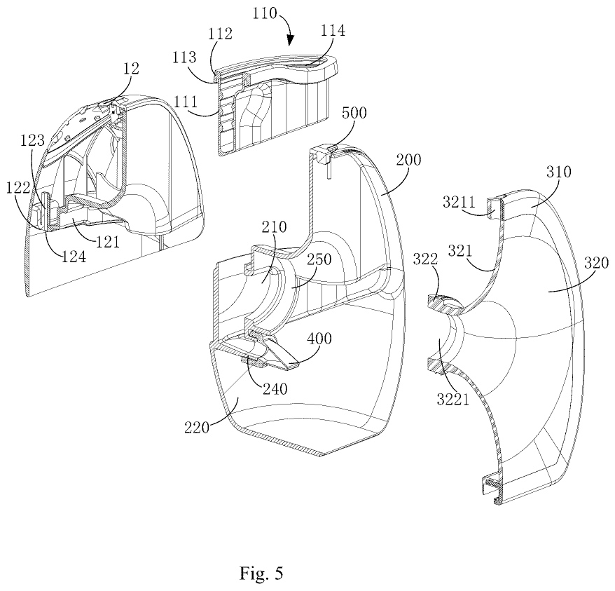

Referring to FIGS. 3, 4 and 5, in this example, the elastic bowl 110 is made of elastic material, so that the elastic bowl 110 can contract and expand. The elastic bowl 110 comprises a main bowl 111 and a flange 112. The main bowl 111 may have a generally truncated cylindrical structure, so that the main bowl 111 forms an accommodation cavity 114 which is actually an open cavity with an opening. The flange 112 is disposed at an opening of the main bowl 111, so that the flange 112 is curved and connected with the main bowl 111, the flange 112 can be disposed around the main bowl 111, and an annular gap 113 is formed between the main bowl 111 and the flange 112. During a mounting process of the elastic bowl 110, the main bowl 111 is accommodated in the storage bin 200, the flange 112 is located outside the storage bin 200, and the storage bin 200 fits with the annular gap 113. Through the fitting between the storage bin 200 and the annular gap 113, the storage bin 200 can support and limit the elastic bowl 110, thereby mounting the elastic bowl 110 on the storage bin 200. In other words, the elastic bowl 110 is hung on the storage bin 200 by the flange 112.

There is an adjustment cavity 210 between the main bowl 111 of the elastic bowl 110 and the storage bin 200, that is, the main bowl 111 and the storage bin 200 define a boundary of the adjustment cavity 210. When the elastic bowl 110 is in a natural state, a volume of the accommodation cavity 114 is the largest, and the elastic bowl 110 occupies the largest volume in the storage bin 200, so that a volume of the adjustment cavity 210 is the smallest. When the elastic bowl 110 completely or substantially contracts under the action of external force, the volume of the accommodation cavity 114 is the smallest and approaches zero, which can be generally understood as that the elastic bowl 110 is deflated, and the elastic bowl 110 occupies the smallest volume in the storage bin 200, so that the adjustment cavity 210 has the largest volume. Since the elastic bowl 110 can have elastic deformation, when the external force is removed, the elastic bowl 110 can gradually expand from the contracting state and return to the natural state. Therefore, when the elastic bowl 110 is in a natural state and the volume of the adjustment cavity 210 is the smallest, air pressure in the adjustment cavity 210 is equal to ambient air pressure; in a case where a total amount of air in the adjustment cavity 210 remains unchanged, when the elastic bowl 110 contracts to cause the volume of the adjustment cavity 210 to gradually increase, the air pressure in the adjustment cavity 210 will be smaller than the ambient air pressure, so that there is negative pressure in the adjustment cavity 210. When the elastic bowl 110 completely or substantially contracts, the volume of the adjustment cavity 210 is the largest, so that the negative pressure within the adjustment cavity 210 is the largest.

Referring to FIGS. 3, 4 and 5, in this example, the suction cover 300 is generally in a trumpet shape. The suction cover 300 comprises a hard cover 310 and a soft cover 320. The soft cover 320 is more flexible than the hard cover 310, so that the soft cover 320 is softer than the hard cover 310. The soft cover 320 comprises a fitting part 321 and a mounting part 322. The fitting part 321 is generally annular, and the mounting part 322 is generally columnar and protrudes from the fitting part 321. The mounting part 322 can be inserted into an insertion hole 250 of the storage bin 200, so that the mounting part 322 forms an interference fit with the insertion hole 250, and the mounting part 322 has a sealing effect on the insertion hole 250. A guide hole 3221 in fluid communication with the adjustment cavity 210 is provided in the mounting part 322. The milk sucked by the breast pump 10 can flow into the adjustment cavity 210 through the guide hole 3221. The entire soft cover 320 and the storage bin 200 can form a storage cavity 220 used to store milk. A periphery of the fitting part 321 abuts the storage bin 200, so that the fitting part 321 has a sealing effect on the storage cavity 220.

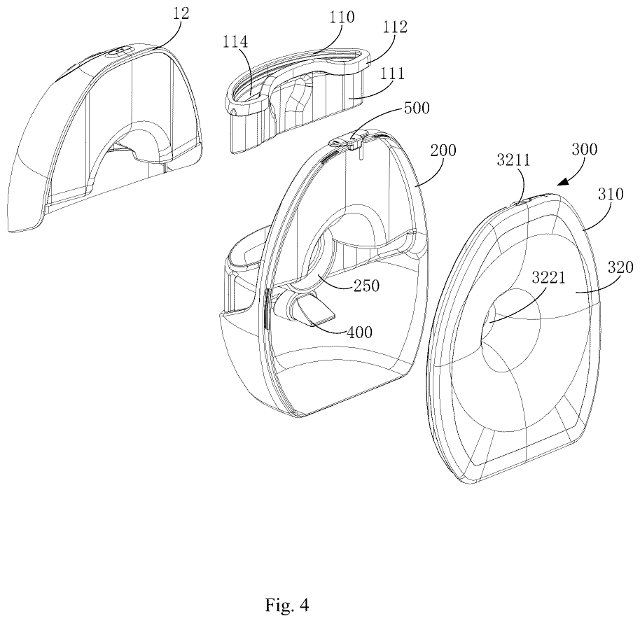

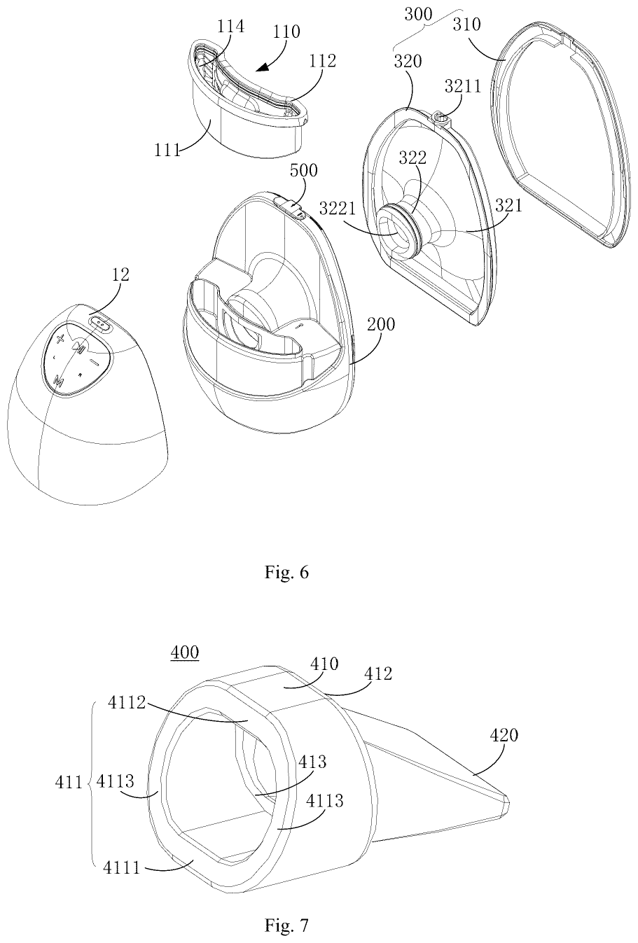

Referring to FIGS. 4, 5 and 6, the fitting part 321 can be provided with a liquid outlet hole 3211 which can connect the outside with the storage cavity 220. The plug 500 and the liquid outlet hole 3211 fit with each other, so that the plug 500 plugs the liquid outlet hole 3211. When the plug 500 is pulled out of the liquid outlet hole 3211, the milk in the storage cavity 220 can flow out of the liquid outlet hole 3211. When the plug 500 plugs the liquid outlet hole 3211, the milk in the storage cavity 220 cannot flow out of the liquid outlet hole 3211.

The soft cover 320 is worn on the breast. Since the soft cover 320 is relatively soft, comfort of contact between the soft cover 320 and the breast can be improved. The soft cover 320 can also massage the breast to a certain extent. The hard cover 310 has relatively large hardness and rigidity. By snap connection between the hard cover 310 and the storage bin 200, stability and reliability of the connection between the entire suction cover 300 and the storage bin 200 can be improved. Therefore, the suction cover 300 uses a combination of soft and hard covers, which improves comfort of wearing the suction cover 300, and also ensures the stability and reliability of the connection between the suction cover 300 and the storage bin 200.

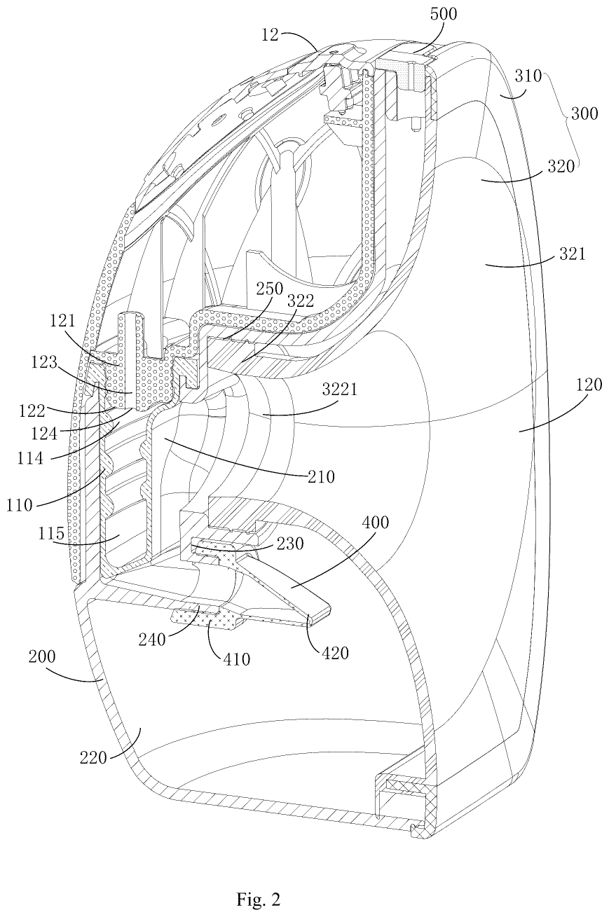

Referring to FIGS. 2, 3 and 4, in this example, the storage bin 200 is integrally formed. The storage bin 200 has a mounting surface 230 which is disposed toward the fitting part 321 of the soft cover 320. When the breast pump 10 is in a working state, the mounting surface 230 extends along the direction of gravity. The storage bin 200 further comprises a flow nozzle 240 that protrudes relative to the mounting surface 230. An angle between an axial direction of the flow nozzle 240 and the direction of gravity is 80° to 90°, so that the axis of the flow nozzle 240 extends generally along the horizontal direction. For example, the angle between the axial direction of the flow nozzle 240 and the direction of gravity may be 90°, so that the axis of the flow nozzle 240 is perpendicular to the mounting surface 230. The angle between the axial direction of the flow nozzle 240 and the direction of gravity may also be 80° or 85°. The control valve 400 is sleeved on the flow nozzle 240, and an inner cavity of the control valve 400 will communicate with the adjustment cavity 210. The flow nozzle 240 and the inner cavity of the control valve 400 can form an interference fit, so that the flow nozzle 240 can fix and support the control valve 400, and both the flow nozzle 240 and the control valve 400 will seal the adjustment cavity 210 and the inner cavity of the control valve 400 at the same time. During the use of the breast pump 10, the control valve 400 often needs to be disassembled from the flow nozzle 240 for washing in order to recycle the control valve 400. Therefore, the control valve 400 must be frequently disassembled and installed. If the axial direction of the flow nozzle 240 extends along the direction of gravity so that the control valve 400 is sleeved on the flow nozzle 240 from bottom to top, which may cause inconvenience in installation and disassembly of the control valve 400, thereby reducing efficiency of installation and disassembly of the control valve 400 and affecting convenience of using the breast pump 10. With respect to the breast pump 10 in the above example, since the angle between the axial direction of the flow nozzle 240 and the direction of gravity is 80° to 90°, the axis of the flow nozzle 240 extends substantially in the horizontal direction, and the control valve 400 can be sleeved on the flow nozzle 240 in the horizontal direction, thus greatly improving the convenience of installation and disassembly of the control valve 400, improving the efficiency of installation and disassembly of the control valve 400, and ultimately improving practical convenience of the breast pump 10. In other examples, the angle between the axial direction of the flow nozzle 240 and the direction of gravity may be less than or equal to 90° regardless of the efficiency of installation and disassembly of the control valve 400.

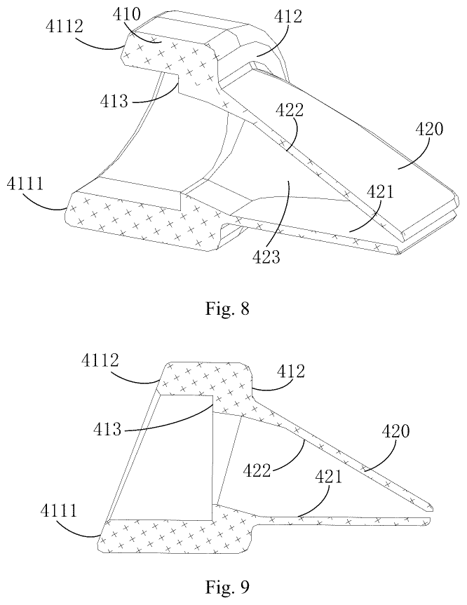

Referring to FIGS. 7, 8 and 9, in this example, the control valve 400 may be a duckbill valve. The control valve 400 comprises a fixed cylinder 410 and an opening and closing cylinder 420 fixed on the fixed cylinder 410. The fixed cylinder 410 has two end surfaces in the axial direction, which are respectively noted as a first end surface 411 and a second end surface 412. When the breast pump 10 is in a working state, the first end surface 411 may be disposed at an acute angle with the direction of gravity, and the second end surface 412 may be disposed perpendicular to the direction of gravity, that is, the first end surface 411 is disposed obliquely, and the second end surface 412 is disposed vertically, so that the first end surface 411 and the second end surface 412 are disposed at an acute angle. Of course, both the first end surface 411 and the second end surface 412 may be a curved surface. The first end surface 411 is an annular surface. The first end surface 411 comprises a first section 4111, a second section 4112 and a connecting section 4113. The number of both the first section 4111 and the second section 4112 is one, and the number of the connecting section 4113 is two. The first section 4111 and the second section 4112 are arranged at intervals. One connecting section 4113 is connected between one end of the first section 4111 and one end of the second section 4112, and the other connecting section 4113 is connected between the other end of the first section 4111 and the other end of the second section 4112. A stop surface 413 is disposed in the fixed cylinder 410 and extends in the direction of gravity.

Referring to FIGS. 2 and 3, when the fixed cylinder 410 is mounted on the flow nozzle 240 in a correct mounting orientation, the fixed cylinder 410 and the entire control valve 400 will be correctly mounted on the flow nozzle 240. At this time, the fixed cylinder 410 will be entirely sleeved on the flow nozzle 240 along its circumferential direction, and an end of the flow nozzle 240 will fit with the stop surface 413, so that the stop surface 413 abut the flow nozzle 240 in the horizontal direction, thereby limiting a position of the fixed cylinder 410 in the horizontal direction. Moreover, the first end surface 411 is closer to the elastic bowl 110 than the stop surface 413, and the second section 4112 corresponds to the mounting surface 230; the first section 4111 is farther from the stop surface 413 than the second section 4112, and the first section 4111 is also farther from the stop surface 413 than the mounting surface 230; and thus a distance from the first section 4111 to the stop surface 413 is greater than distances from the second section 4112 and the mounting surface 230 to the stop surface 413. The second section 4112 may abut the mounting surface 230 or may be spaced apart from the mounting surface 230. Therefore, the distance from the mounting surface 230 to the stop surface 413 is greater than or equal to the distance from the second section 4112 to the stop surface 413.

If the fixed cylinder 410 is rotated 180° from the correct mounting orientation to a wrong mounting orientation, the fixed cylinder 410 and the entire control valve 400 will be wrongly mounted on the flow nozzle 240. During the wrong mounting process, the first section 4111 will correspond to the mounting surface 230. Since the distance between the first section 4111 and the stop surface 413 is greater than the distance between the second section 4112 and the stop surface 413, even if the first section 4111 abuts the mounting surface 230, due to interference of the mounting surface 230, on the one hand, an end of the flow nozzle 240 cannot abut with the stop surface 413; on the other hand, the fixed cylinder 410 cannot be entirely sleeved on the flow nozzle 240 along its circumferential direction, that is, a part of the fixed cylinder 410 corresponding to the second section 4112 cannot contact the flow nozzle 240 or be sleeved on the flow nozzle 240, so that the fixed cylinder 410 and the entire control valve 400 cannot be mounted on the flow nozzle 240, thereby warning an operator that the fixed cylinder 410 is mounted wrongly, so as to remind the operator to mount the fixed cylinder 410 on the flow nozzle 240 in the correct mounting orientation.

Therefore, a special structure of the above fixed cylinder 410 can provide a warning that the control valve 400 is wrongly mounted, and can also be understood as playing a “foolproof” role to ensure that the fixed cylinder 410 can only be mounted correctly on the flow nozzle 240 in the correct mounting orientation.

Referring to FIGS. 7, 8 and 9, in this example, the opening and closing cylinder 420 is protrudingly disposed on the second end surface 412 of the fixed cylinder 410, and the inner cavities of the opening and closing cylinder 420 and the fixed cylinder 410 communicate with each other and together form the inner cavity of the entire control valve 400. The opening and closing cylinder 420 each has a first inner surface 421, a second inner surface 422 and a connecting inner surface 423. The number of the first inner surface 421 and the second inner surface 422 is one, and the number of the connecting inner surface 423 is two. The first inner surface 421 and the second inner surface 422 may be flat and spaced apart from each other, and the connecting inner surface 423 may be a curved surface. One connecting inner surface 423 is connected between one end of the first inner surface 421 and one end of the second inner surface 422, the other connecting inner surface 423 is connected between the other end of the first inner surface 421 and the other end of the second inner surface 422. When the opening and closing cylinder 420 contracts or expands and the connecting inner surface 423 also contracts or expands, the first inner surface 421 and the second inner surface 422 move close to or away from each other. When the first inner surface 421 and the second inner surface 422 move close to each other until completely or substantially fitting, the inner cavity of the opening and closing cylinder 420 almost disappears. At this time, the control valve 400 is closed, so that the storage cavity 220 cannot communicate with the adjustment cavity 210 through the inner cavity of the control valve 400, that is, the storage cavity 220 and the adjustment cavity 210 are disconnected and isolated from each other. When the first inner surface 421 and the second inner surface 422 are separated from each other at a certain distance, a volume of the inner cavity of the opening and closing cylinder 420 increases. At this time, the control valve 400 is opened, so that the storage cavity 220 is in fluid communication with the adjustment cavity 210 through the inner cavity of the control valve 400.

Referring to FIGS. 2 and 3, in this example, the first inner surface 421 is disposed close to the first section 4111, and the second inner surface 422 is disposed close to the second section 4112. The first inner surface 421 may extend along the horizontal direction and be disposed horizontally, the second inner surface 422 is inclined at an angle with the first inner surface 421 along a direction where the fixed cylinder 410 points to the opening and closing cylinder 420, so that a distance between the first inner surface 421 and the second inner surface 422 is reduced. When the control valve 400 is mounted correctly and the breast pump 10 is in a working state, the second inner surface 422 is located above the first inner surface 421. In a case where the control valve 400 is mounted correctly, when the control valve 400 is opened, milk in the adjustment cavity can flow into the storage cavity 220 through the control valve 400. Since the control valve 400 is disposed horizontally and the second inner surface 422 is inclined, it can be ensured that the milk quickly flows from the opening and closing cylinder 420 into the storage cavity 220 according to basic principles of fluid dynamics.

If the control valve 400 is not “foolproof” and the control valve 400 is mounted wrongly, the first inner surface 421 will be located above the second inner surface 422, so that the milk in the control valve 400 cannot flow into the storage cavity 220 quickly. Therefore, the control valve 400 is designed to be “foolproof”. When the control valve 400 is mounted, it can be ensured that the second inner surface 422 is generally located above the first inner surface 421, thereby allowing milk to quickly flow from the opening and closing cylinder 420 into the storage cavity 220.

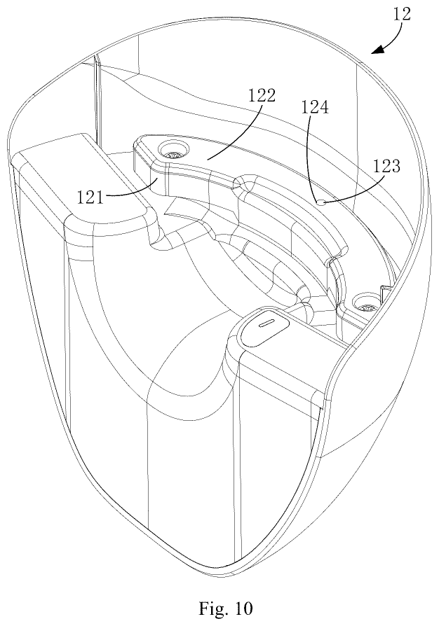

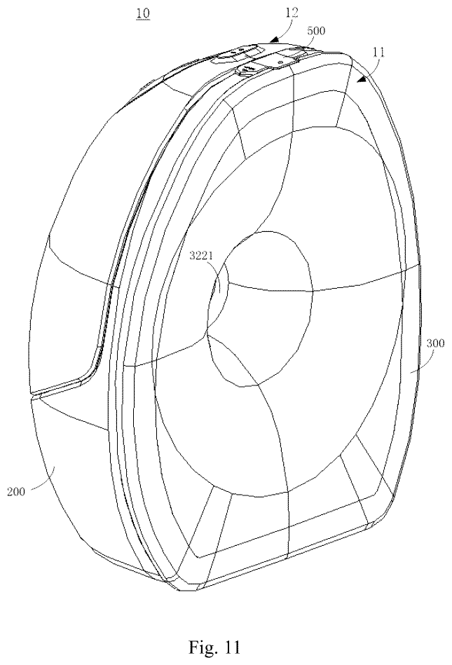

Referring to FIGS. 2, 3 and 10, in this example, the main body 12 comprises a boss 121 that has a solid structure. When the main body 12 is mounted on the storage bin 200, the boss 121 can form an interference fit with the accommodation cavity 114 of the elastic bowl 110, thereby sealing the accommodation cavity 114. Since part of space of the accommodation cavity 114 is filled by the boss 121, the remaining unfilled space of the accommodation cavity 114 is an effective space 115 of the accommodation cavity 114. The boss 121 has a limiting surface 122 which is disposed toward a bottom of the elastic bowl 110. A suction hole 123 is provided in the boss 121 and penetrates the limiting surface 122. Therefore, the suction hole 123 has an opening on the limiting surface 122, which is noted as an extraction opening 124. A diameter of the extraction opening 124 is 1 mm to 3 mm. It can be understood that the diameter of the extraction opening 124 is about 2 mm. For example, the diameter of the extraction opening 124 may be 1 mm, 2 mm, or 3 mm. When an air pump of the main body 12 exhausts gas in the effective space 115 of the accommodation cavity 114 through the suction hole 123, a volume of the effective space 115 will approach zero, so that the main bowl 111 contracts and covers the limiting surface 122. When the air pump of the main body 12 stops extracting air, the volume of the effective space 115 returns to an original state.

Referring to FIGS. 2 and 3, during use, the suction cover 300 is worn on the breast. At this time, both the elastic bowl 110 and the control valve 400 are in a natural state, the control valve 400 is opened, the adjustment cavity 210 communicates with the storage cavity 220 through the inner cavity of the control valve 400, and air pressure of the adjustment cavity 210 and the inner cavity of the control valve 400 is the ambient air pressure. When the air pump of the main body 12 exhausts the gas in the effective space 115 of the accommodation cavity 114 through the suction hole 123, the elastic bowl 110 contracts, and the volume of the effective space 115 is approximately zero, and the occupied volume of the elastic bowl 110 in the storage bin 200 is the smallest, so that the volume of the adjustment cavity 210 is the largest. Therefore, the adjustment cavity 210 and the inner cavity of the control valve 400 will generate negative pressure. Under the action of the negative pressure, on the one hand, the milk of the breast flows into the adjustment cavity 210 through the guide hole 3221 on the suction cover 300, thereby realizing suction of human milk by the breast pump 10; on the other hand, under the action of the negative pressure, the control valve 400 causes the first inner surface 421 and the second inner surface 422 of the opening and closing cylinder 420 fit with each other, the control valve 400 is closed, the adjustment cavity 210 and the storage cavity 220 are isolated from each other, and the milk in the adjustment cavity 210 cannot flow into the storage cavity 220 through the control valve 400. Therefore, through the closing of the control valve 400, during the suction of milk, the adjustment cavity 210 cannot communicate with the storage cavity 220, ensuring that sufficient negative pressure is generated in the adjustment cavity 210 and the guide hole 3221, so that the breast pump 10 can suck more milk in a unit time.

When the air pump stops extracting air through the suction hole 123, the elastic bowl 110 stretches and returns to an original state, the effective space 115 of the accommodation cavity 114 returns to an original state, and the volume of the adjustment cavity 210 is the smallest, so that the negative pressure in the adjustment cavity 210 and the guide hole 3221 disappears, which makes the first inner surface 421 and the second inner surface 422 of the opening and closing cylinder 420 away from each other. The control valve 400 is opened, the adjustment cavity 210 and the storage cavity 220 are in fluid communication with each other, and the milk in the adjustment cavity 210 will flow into the storage cavity 220 through the control valve 400, thereby storing milk in the storage cavity 220. Therefore, the air pump periodically evacuates the accommodation cavity 114 through the suction hole 123, which can ensure that the suction cover 300 sucks milk and stores the milk in the storage cavity 220. The volume of the effective space 115 of the accommodation cavity 114 has a direct impact on the generation of negative pressure. The larger the volume of the effective space 115, the greater the negative pressure, so the negative pressure is proportional to the volume of the effective space 115.

If a sink hole with a large diameter is formed on the limiting surface 122 of the boss 121, the boss 121 has a hollow structure, and then a suction hole 123 is provided on a bottom wall of the sink hole. The sink hole directly communicates with the effective space 115 of the accommodation cavity 114 and has a diameter much larger than the diameter of the suction hole 123. When the air pump exhausts the gas in the effective space 115 of the accommodation cavity 114 through the suction hole 123, the main bowl 111 of the elastic bowl 110 will adhere to the limiting surface 122, but the main bowl 111 cannot further enter the sink hole, so that the main bowl 111 cannot fill space of the sink hole, and there is still a sink hole between the elastic bowl 110 after contraction and the boss 121, that is, there is a large gap between the elastic bowl 110 after contraction and the boss 121.

Therefore, during the process of extracting air, the gas in the effective space 115 and the sink hole may be exhausted. When the gas in the effective space 115 is exhausted, the effective space 115 disappears and is released, and the increased volume of the adjustment cavity 210 may be equal to the released volume of the effective space 115, and the increased volume of the adjustment cavity 210 will directly contribute to the negative pressure. However, when the gas in the sink hole is exhausted, the main bowl 111 cannot be further deformed and continues to fill into the sink hole, so that the volume in the sink hole cannot be released. Therefore, the increased volume of the adjustment cavity 210 may be equal to the volume of the effective space 115, but cannot be equal to the sum of the volumes of the effective space 115 and the sink hole, so that extraction of gas in the sink hole does not contribute to generation of negative pressure in the adjustment cavity 210. Therefore, extraction of gas in the effective space 115 by the air pump can generate negative pressure; but extraction of gas in the sink hole by the air pump cannot generate negative pressure; so that extraction of the gas in the sink hole by the air pump is idle work, which will reduce a utilization rate of energy by the air pump and the entire breast pump 10.

With regard to the breast pump 10 in the above example, the boss 121 has a solid structure. When gas is extracted at the suction hole 123 to cause the main bowl 111 to completely or substantially contract, the main bowl 111 will tightly adhere to the limiting surface 122, so that a gap between the main bowl 111 and the boss 121 disappears and is approximately zero. It can be understood that there is no gap between the main bowl 111 and the boss 121, and the increased volume of the adjustment cavity 210 is equal to the volume of the effective space 115. Energy of extracting air in the effective space 115 by the air pump is all converted into negative pressure, substantially eliminating the air pump from extracting air in the gap between the main bowl 111 and the boss 121, which cannot contribute to the negative pressure, thereby substantially eliminating the idle work of the air pump and improving the utilization rate of energy by the air pump and the entire breast pump 10. As the energy utilization rate is improved, when output power of the air pump is the same, the breast pump 10 can suck more milk per unit time.

The elastic bowl 110, the storage bin 200, the suction cover 300, the control valve 400 and the plug 500 can all be made of a food-grade material that can withstand high-temperature boiling. When the main body 12 is disassembled from the storage bin 200, the elastic bowl 110, the suction cover 300, the control valve 400 and the plug 500 can all be disassembled from the storage bin 200, so that the elastic bowl 110, the suction cover 300, the control valve 400, the plug 500 and the storage bin 200 are boiling sterilized. The storage bin 200 is integrally formed and has a modular design, which can improve the convenience of disassembly and assembly of the storage bin 200. The storage bin 200 can be polished to improve leakproof-ness of assembly positions of the elastic bowl 110, the control valve 400 and the suction cover 300, and to improve the energy utilization rate of the air pump and the breast pump 10.

### SECOND EXAMPLE

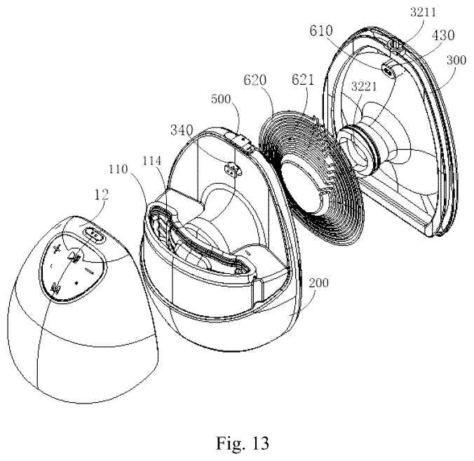

Referring to FIGS. 11, 12 and 13, the breast pump 10 provided in the second example of the present disclosure has a wearable structure, that is, the breast pump 10 can be directly worn on the human breast to suck milk. The breast pump 10 comprises a suction mechanism 11 worn on the breast and a main body 12 disposed on the suction mechanism 11. The suction mechanism 11 comprises an elastic bowl 110, a storage bin 200, a suction cover 300, a control valve 400 and a plug 500. The main body 12, the elastic bowl 110, the suction cover 300, the control valve 400 and the plug 500 can all be disposed on the storage bin 200 by detachable connection.

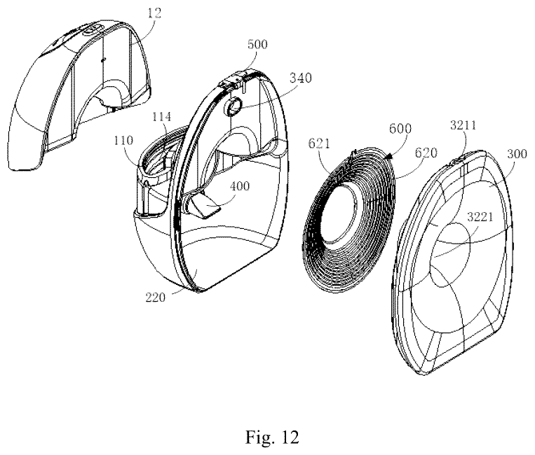

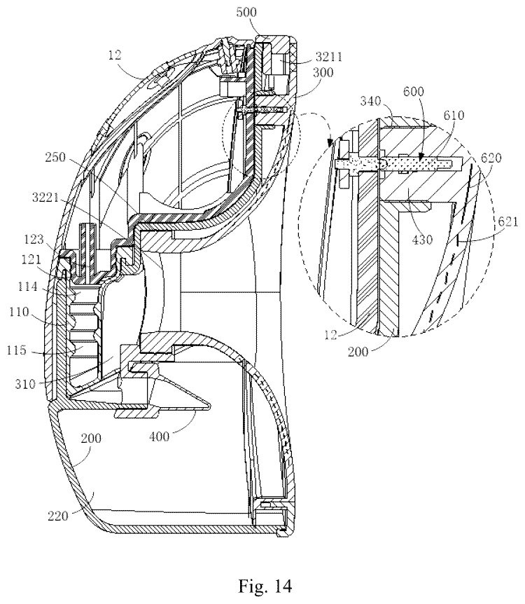

Referring to FIGS. 12, 13 and 14, the elastic bowl 110 is made of elastic material, so that the elastic bowl 110 can contract and expand. The elastic bowl 110 forms an accommodation cavity 114 which is actually an open cavity with an opening. The elastic bowl 110 is fixed on the storage bin 200 and is at least partially accommodated in the storage bin 200. A part of the elastic bowl 110 that forms the accommodation cavity 114 is accommodated in the storage bin 200. There is an adjustment cavity 210 between the elastic bowl 110 and the storage bin 200, that is, the elastic bowl 110 and the storage bin 200 define the boundary of the adjustment cavity 210. When the elastic bowl 110 is in a natural state, the volume of the accommodation cavity 114 of the elastic bowl 110 is the largest, and the elastic bowl 110 occupies the largest volume in the storage bin 200, so that the volume of the adjustment cavity 210 is the smallest. When the elastic bowl 110 completely or substantially contracts under the action of external force, the volume of the accommodation cavity 114 is the smallest and approaches zero, which can be generally understood that the elastic bowl 110 is deflated, and the volume occupied by the elastic bowl 110 in the storage bin 200 is the smallest, so that the adjustment cavity 210 has the largest volume. Since the elastic bowl 110 can be elastically deformed, when the external force is removed, the elastic bowl 110 can gradually expand from the contracting state and return to the natural state. Therefore, when the elastic bowl 110 is in the natural state and the volume of the adjustment cavity 210 is the smallest, air pressure in the adjustment cavity 210 is equal to the ambient air pressure; when the total amount of air in the adjustment cavity 210 remains unchanged, the elastic bowl 110 contracts, so that when the volume of the adjustment cavity 210 gradually increases, the air pressure in the adjustment cavity 210 will be smaller than the ambient air pressure, so that there is negative pressure in the adjustment cavity 210. When the elastic bowl 110 completely or substantially contracts, the volume of the adjustment cavity 210 is the largest, so that the negative pressure within the adjustment cavity 210 is the largest.

Referring to FIGS. 12, 13 and 14, the suction cover 300 is generally in a trumpet shape and is worn on the human breast. The middle part of the suction cover 300 is inserted into an insertion hole 250 of the storage bin 200. The suction cover 300 and the insertion hole 250 forms an interference fit, which has a sealing effect on the insertion hole 250. An edge part of the suction cover 300 may be snap-connected with the storage bin 200, thereby fixing the suction cover 300. A guide hole 3221 is provided in the suction cover 300 and is connected with the adjustment cavity 210. The milk sucked by the breast pump 10 can flow into the adjustment cavity 210 through the guide hole 3221. The suction cover 300 and the storage bin 200 may form a storage cavity 220, and the storage cavity 220 is used to store milk. The suction cover 300 is also provided with a liquid outlet hole 3211 which can communicate with the outside and the storage cavity 220. The plug 500 fits with the liquid outlet hole 3211, so that the plug 500 plugs the liquid outlet hole 3211. When the plug 500 is pulled out of the liquid outlet hole 3211, the milk in the storage cavity 220 can flow out of the liquid outlet hole 3211. When the plug 500 plugs the liquid outlet hole 3211, the milk in the storage cavity 220 cannot flow out of the liquid outlet hole 3211.

Referring to FIGS. 12, 13 and 14, the storage bin 200 may be integrally formed. The control valve 400 is sleeved on the storage bin 200. The inner cavity of the control valve 400 will communicate with the adjustment cavity 210. The control valve 400 may be a duckbill valve and produce compression and expansion. When the control valve 400 is compressed under the action of external force, an end of the control valve 400 close to the storage cavity 220 is closed due to compression. At this time, the control valve 400 is closed, so that the adjustment cavity 210 and the storage cavity 220 are disconnected and isolated from each other. When the external force is removed, the control valve 400 returns to the original state, and an end of the control valve 400 close to the storage cavity 220 is opened. At this time, the control valve 400 is opened, so that the adjustment cavity 210 can communicate with the inner cavity of the control valve 400 for storage.

In particular, referring to FIGS. 12, 13 and 14, what is different from the first example of the present disclosure is that the breast pump 10 in the second example may further comprise a heating element 600 and a temperature sensor. The heating element 600 is disposed on the suction cover 300 and is electrically connected with the main body 12, so that a battery of the main body 12 supplies power to the heating element 600, and the heating element 600 converts electric energy into thermal energy. When the breast pump 10 sucks milk, the heating element 600 generates heat. A temperature of the heating element 600 may be higher than body temperature. The heat of the heating element 600 is conducted to the breast, and can dredge breast ducts, increase the amount of milk produced by the breast, and thus increase the amount of milk sucked by the breast pump 10 per unit time. The heat of the heating element 600 can also improve comfort, reduce pain caused by the breast during a milk discharge process, and alleviate milk blockage of pregnant women during lactation. The temperature sensor is disposed on the heating element 600 and connected with a controller in the main body 12. The temperature sensor may feedback the temperature information to the controller, and the controller may adjust output power of the battery to the heating element 600, and thus can more accurately control and adjust the temperature of the heating element 600, so that the heat generated by the heating element 600 can meet needs of different users. Therefore, the breast pump 10 can generate heat by itself, and there is no need to set other heat sources outside the breast pump 10, thereby improving convenience of using the breast pump 10.

Referring to FIGS. 12, 13 and 14, in the second example, the heating element 600 is embedded in the suction cover 300. For example, the heating element 600 can be embedded in the suction cover 300 by in-mold injection molding. The heating element 600 and the suction cover 300 form a non-disassemblable connection relationship. The heating element 600 comprises a heating part 620 and a conductive part 610 which are connected with each other. The heating part 620 is used to convert electric energy into thermal energy to generate heat. The conductive part 610 is used to transmit the electric energy of the battery to the heating part 620. The conductive part 620 is electrically conductive. Resistance of the conductive part 610 may be smaller than resistance of the heating part 620, so that the conductive part 610 has excellent conductive function. The heating part 620 may be a spiral resistance wire 621, and, may be an infrared lamp, a flexible heating sheet, a graphene heating film, or the like. The conductive part 610 may be a pin with a columnar structure. The suction cover 300 comprises a protruding column 430 which is protrudingly disposed on the main body of the suction cover 300. When the breast pump 10 is in a working state, an extending direction of the protruding column 430 may be perpendicular to the direction of gravity, so that the protruding column 430 extends in the horizontal direction. The conductive part 610 penetrates the protruding column 430, and the conductive part 610 also extends in the horizontal direction perpendicular to the direction of gravity. An end of the conductive part 610 away from the heating part 620 may be exposed outside the protruding column 430. When the protruding column 430 is inserted into a through hole 340 of the storage bin 200, the conductive part 610 will abut the pin on the main body 12 in the horizontal direction, so that the battery on the main body 12 supplies power to the heating element 600. When the heating element 600 generates heat, the heat generated by the heating element 600 is conducted to the breast through the suction cover 300. The protruding column 430 may form an interference fit with the through hole 340 of the storage bin 200 to prevent milk from leaking through the through hole 340 and prevent the milk from corroding the main body 12.

In another mode of the second example of the present disclosure, the storage bin 200, the elastic bowl 110, the suction cover 300, the control valve 400 and the plug 500 may all be made of a food-grade material that can withstand high-temperature boiling. When the main body 12 is disassembled from the storage bin 200, the elastic bowl 110, the suction cover 300, the control valve 400 and the plug 500 may all be disassembled from the storage bin 200 to boiling sterilize the elastic bowl 110, the suction cover 300, the control valve 400 and the plug 500. The storage bin 200 is integrally formed and has a modular design, which can improve the convenience of disassembly and assembly of the storage bin 200. The storage bin 200 can be polished to improve leakproofness of assembly positions of the elastic bowl 110, the control valve 400 and the suction cover 300, and to improve the energy utilization rate of the air pump and the breast pump 10.

Referring to FIGS. 12, 13 and 14, in another mode of the second example of the present disclosure, the main body 12 comprises a boss 121, and the boss 121 can form an interference fit with the accommodation cavity 114 of the elastic bowl 110, thereby sealing the accommodation cavity 114. Since part of space of the accommodation cavity 114 is filled by the boss 121, the remaining unfilled space of the accommodation cavity 114 is an effective space 115 of the accommodation cavity 114. A suction hole 123 is provided in the boss 121, and a diameter of the suction hole 123 is 1 mm to 3 mm. It can be understood that the diameter of the suction hole 123 is about 2 mm. For example, the diameter of the suction hole 123 may be 1 mm, 2 mm, or 3 mm. When an air pump of the main body 12 exhausts gas in the effective space 115 of the accommodation cavity 114 through the suction hole 123, a volume of the effective space 115 will approach zero. When the air pump of the main body 12 stops extracting air, the volume of the effective space 115 returns to an original state.

Referring to FIGS. 12, 13 and 14, during use, the suction cover 300 is worn on the breast. At this time, both the elastic bowl 110 and the control valve 400 are in a natural state, the control valve 400 is opened, the adjustment cavity 210 communicates with the storage cavity 220 through the inner cavity of the control valve 400, and air pressure of the adjustment cavity 210 and the inner cavity of the control valve 400 is the ambient air pressure. When the air pump of the main body 12 exhausts the gas in the effective space 115 of the accommodation cavity 114 through the suction hole 123, the elastic bowl 110 contracts, and the volume of the effective space 115 is approximately zero, and the occupied volume of the elastic bowl 110 in the storage bin 200 is the smallest, so that the volume of the adjustment cavity 210 is the largest. Therefore, the adjustment cavity 210 and the inner cavity of the control valve 400 will generate negative pressure. Under the action of the negative pressure, on the one hand, the milk of the breast flows into the adjustment cavity 210 through the guide hole 3221 on the suction cover 300, thereby realizing suction of human milk by the breast pump 10; on the other hand, the control valve 400 contracts under the action of the negative pressure and is closed, the adjustment cavity 210 and the storage cavity 220 are isolated from each other, and the milk in the adjustment cavity 210 cannot flow into the storage cavity 220 through the control valve 400. Therefore, through the closing of the control valve 400, during the suction of milk, the adjustment cavity 210 cannot communicate with the storage cavity 220, ensuring that sufficient negative pressure is generated in the adjustment cavity 210 and the guide hole 3221, so that the breast pump 10 can suck more milk in a unit time.

When the air pump stops extracting air through the suction hole 123, the elastic bowl 110 stretches and returns to an original state, the effective space 115 of the accommodation cavity 114 returns to an original state, and the volume of the adjustment cavity 210 is the smallest, so that the negative pressure in the adjustment cavity 210 and the guide hole 3221 disappears, which causes the control valve 400 to return to the original state. The control valve 400 is opened, the adjustment cavity 210 and the storage cavity 220 are in fluid communication with each other, and the milk in the adjustment cavity 210 will flow into the storage cavity 220 through the control valve 400, thereby storing milk in the storage cavity 220. Therefore, the air pump periodically evacuates the accommodation cavity 114 through the suction hole 123, which can ensure that the breast pump 10 sucks milk and stores the milk in the storage cavity 220. The volume of the effective space 115 of the accommodation cavity 114 has a direct impact on the generation of negative pressure. The larger the volume of the effective space 115, the greater the negative pressure, so the negative pressure is proportional to the volume of the effective space 115.

### THIRD EXAMPLE

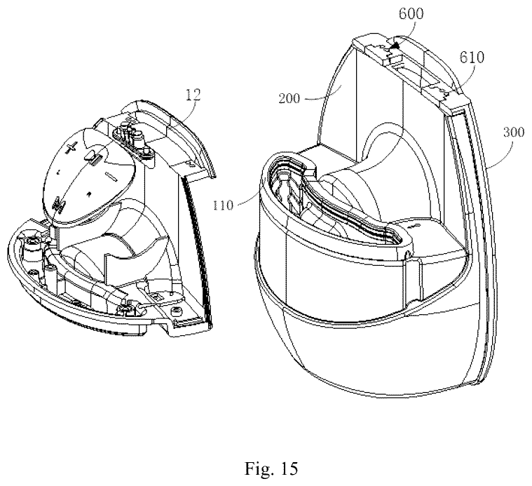

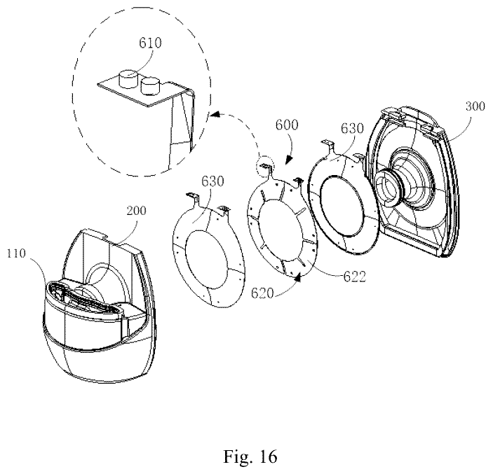

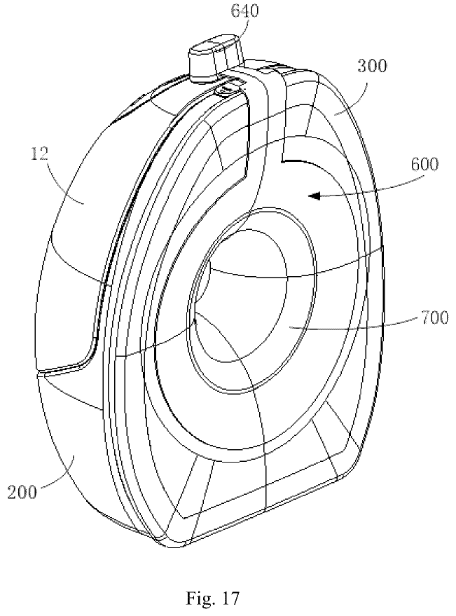

Referring to FIGS. 15 and 16, in the third example, the heating element 600 is also embedded in the suction cover 300. For example, the heating element 600 can be embedded in the suction cover 300 by in-mold injection molding. The heating element 600 and the suction cover 300 form a non-detachable connection relationship. The heating element 600 comprises a heating part 620 and a conductive part 610 which are connected with each other. The heating part 620 is used to convert electric energy into thermal energy to generate heat. The conductive part 610 is used to transmit the electric energy of the battery to the heating part 620. The conductive part 620 is electrically conductive. Resistance of the conductive part 610 may be smaller than resistance of the heating part 620, so that the conductive part 610 has excellent conductive function. The heating part 620 may be substantially annular and is a flexible heating sheet. For example, the heating part 620 may be an FPC heating sheet. The conductive part 610 may be a pin with a columnar structure. When the breast pump 10 is in a working state, the conductive part 610 will extend in the direction of gravity; when the main body 12 is mounted on the storage bin 200, the conductive part 610 will abut the pin on the main body 12 in the direction of gravity, so that the battery on the main body 12 supplies power to the heating element 600.

Since the heating part 620 is a sheet-shaped flexible heating sheet 622, the heating element 600 may further comprise a heat conducting sheet 630 which has a high heat conductivity coefficient, so that the heat conducting sheet 630 has excellent heat conductivity. The number of the heat conducting sheet 630 may be two, and the two heat conducting sheets 630 respectively adhere to two surfaces of the heating part 620 in the thickness direction, so that the heating part 620 is sandwiched between the two heat conducting sheets 630. The heat conducting sheet 630 can, on the one hand, limit a position of the heating part 620, and on the other hand, can evenly transfer heat generated by the heating part, so that the heat generated by the heating part is evenly transferred to the suction cover 300 through the heat conducting sheet 630, thus the heat generated by the suction cover 300 is evenly transferred to the breast, and ultimately heat is evenly distributed on the breast, avoiding discomfort caused by excessive local temperature.

### FOURTH EXAMPLE

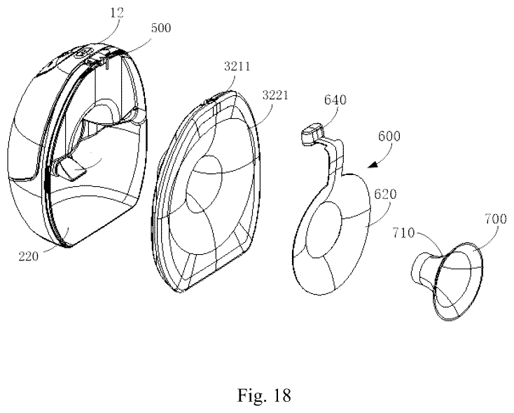

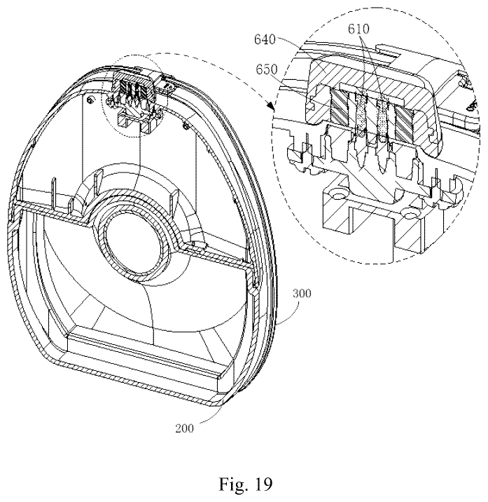

Referring to FIGS. 17, 18 and 19, in the fourth example, the heating element 600 is disposed outside the suction cover 300, and the heating element 600 and the suction cover 300 are detachably connected. Of course, the heating element 600 and the suction cover 300 may also form non-detachable connection. The heat generated by the heating element 600 can be directly conducted to the breast, thereby shortening the transfer path to reduce heat loss, and also allowing the heating element 600 to quickly warm the breast in a short period. When the heating element 600 is damaged, the heating element 600 can also be disassembled from the suction cover 300 for replacement.

The heating element 600 comprises a conductive part 610, a heating part 620, a fixing base 640 and a magnetic element 650. The heating part 620 may be an annular sheet structure and is attached to an outer surface of the suction cover 300. For example, the heating part 620 may be attached to an outer surface of the suction cover 300 which is in direct contact with the breast, and thus the heating part 620 is in direct contact with the breast, so that the heat generated by the heating part 620 is directly conducted to the breast. The magnetic element 650 is disposed in the fixing base 640. When the fixing base 640 is placed on the main body 12, the magnetic element 650 will generate magnetic attraction force with the main body 12, so that the fixing base 640 is fixed on the main body 12 by detachable connection through the magnetic attraction force. The fixing base 640 may also be fixed on the main body 12 by detachable connection such as snap connection. The conductive part 610 may be a pin with a columnar structure and penetrates the fixing base 640. When the breast pump 10 is working, the conductive part 610 may extend along the direction of gravity. When the mounting base 640 is fixed on the main body 12, the conductive part 610 abuts the pin on the main body 12 along the direction of gravity, so that the battery of the main body 12 supplies power to the conductive part 610 and the heating part 620.

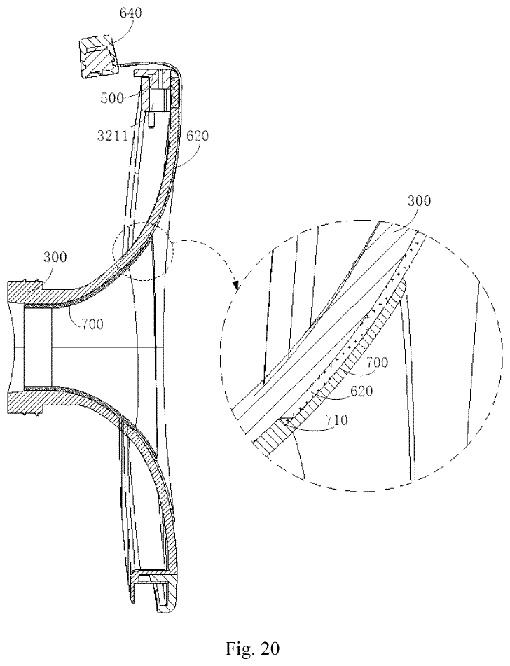

Referring to FIG. 20, the breast pump 10 may further comprise an adjustment plug 700 inserted into the suction cover 300, which may be partially covered by the heating part 620. The adjustment plug 700 is provided with an annular step surface 710, and the heating part 620 abuts the step surface 710. By providing the adjustment plug 700, the heating part 620 may be sandwiched between the suction cover 300 and the adjustment plug 700, thereby improving mounting stability of the heating part 620. At the same time, the step surface 710 limits a position of the heating sheet. Moreover, the adjustment plug 700 may be replaced according to the size of nipple, thereby further improving comfort of using the breast pump 10.

### FIFTH EXAMPLE

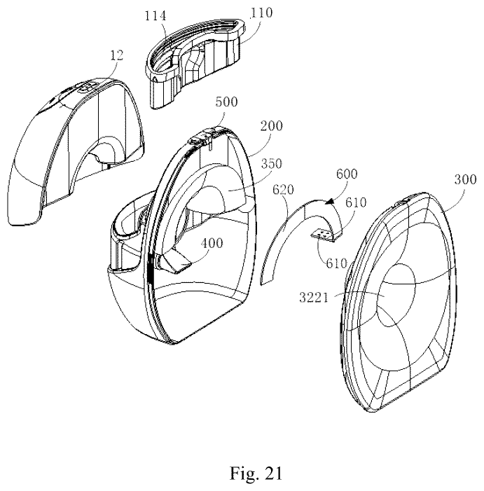

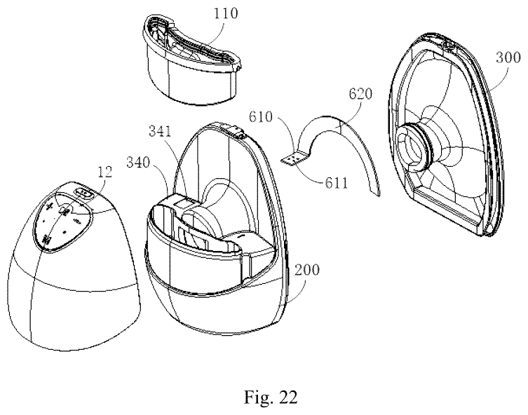

Referring to FIGS. 21 and 22, the breast pump 10 may further comprise the heating element 600 and a temperature sensor. Different from the solution of the second to fourth examples, the heating element 600 in the fifth example of the present disclosure is disposed on the storage bin 200. It can be understood that the heating element 600 may be disposed on at least one of the storage bin 200 and the main body 12. For example, the heating element 600 is disposed only on the main body 12. For another example, the heating element 600 is disposed on both the storage bin 200 and the main body 12. The battery of the main body 12 supplies power to the heating element 600, and the heating element 600 converts electric energy into thermal energy. When the breast pump 10 sucks milk, the heating element 600 generates heat and has a temperature. The temperature of the heating element 600 may be higher than body temperature. The heat of the heating element 600 is conducted to the breast and can dredge breast ducts, increase the amount of milk produced by the breast, and thus increase the amount of milk sucked by the breast pump 10 per unit time. The heat of the heating element 600 can also improve comfort, reduce pain caused by the breast during a milk discharge process, and alleviate milk blockage of pregnant women during lactation. The temperature sensor is disposed on the heating element 600 and connected with a controller in the main body 12. The temperature sensor may feedback the temperature information to the controller, and the controller may adjust output power of the battery to the heating element 600, and thus can accurately control and adjust the temperature of the heating element 600, so that the heat generated by the heating element 600 can meet needs of different users. Therefore, the breast pump 10 can generate heat by itself, and there is no need to set other heat sources outside the breast pump 10, thereby improving convenience of using the breast pump 10.

Referring to FIGS. 21 and 22, in the fifth example, the heating element 600 is disposed on the storage bin 200. For example, the heating element 600 may be embedded in the storage bin 200 by in-mold injection molding, so that the heating element 600 and the storage bin 200 form non-detachable connection. The heating element 600 comprises a heating part 620 and a conductive part 610 which are connected with each other. The heating part 620 may be a heating material such as an infrared lamp, a resistance wire, a flexible heating sheet, or a graphene heating film. Resistivity of the conductive part 610 may be low, so that the conductive part 610 has excellent conductive performance, and the heating part 620 converts electric energy into heat. The storage bin 200 comprises a protrusion 350 that protrudes toward the suction cover 400 relative to a main body part of the storage bin 200. The heating part 620 is disposed in the protrusion 350 which abuts the suction cover 400, so that the heating part 620 approaches the suction cover 400 as much as possible. When the heating part 620 generates heat, a heat transfer path of the heating part 620 can be shortened, so that the heat generated by the heating part 620 can be quickly conducted to the human breast through the protrusion 350 and the suction cover 400, thereby improving a heat conduction speed of the heating part 620, reducing loss during heat transfer, and improving a utilization rate of energy.

The storage bin 200 has a bearing surface 345. When the breast pump 10 is in a working state, the bearing surface 345 is perpendicular to the direction of gravity, so that the bearing surface 345 is a plane along the horizontal direction. When the main body 12 is mounted on the storage bin 200, the bearing surface 345 can support the main body 12 along the direction of gravity. The bearing surface 345 may be provided with a sink hole 341, the conductive part 610 may be at least partially accommodated in the sink hole 341, and the surface of the conductive part 610 may be aligned with the bearing surface 345. The conductive part 610 is provided with a contact 611. When the main body 12 is mounted on the storage bin 200, the pin on the main body 12 can touch the contact 611, thus realizing electrical connection between the conductive part 610 and the main body 12, so that the battery of the main body 12 supplies power to the heating part 620 through the conductive part 610.

### SIXTH EXAMPLE

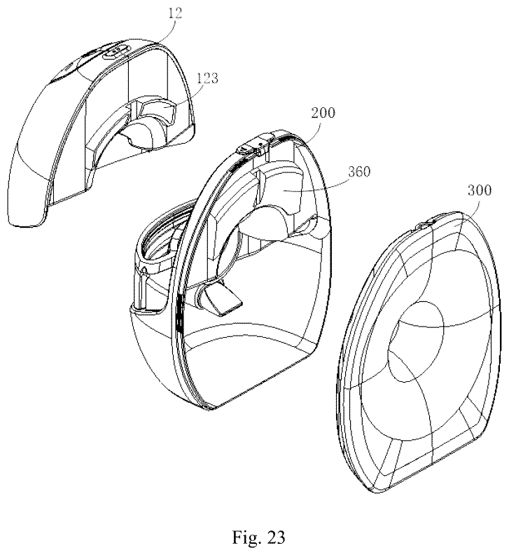

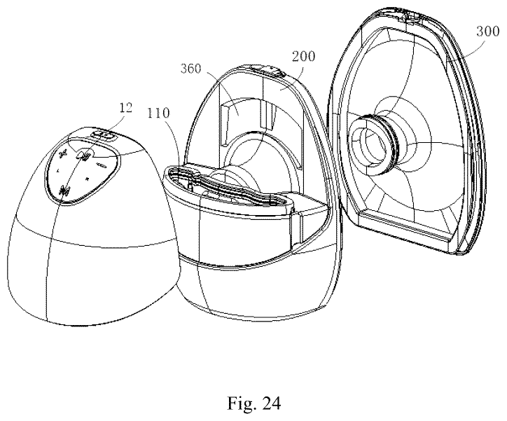

Referring to FIGS. 23 and 24, in the sixth example, the heating element 600 is disposed on the main body 12. For example, the heating element 600 may be embedded in the main body 12 through in-mold injection molding, so that the heating element 600 and the main body 12 form non-detachable connection. The heating element 600 comprises a heating part 620 which may be an infrared lamp, a resistance wire, a flexible heating sheet, a graphene heating film, or the like. The main body 12 comprises a first bulging part 123 that bulges toward the storage bin 200. The storage bin 200 comprises a second bulging part 360 that bulges toward the suction cover 400. The heating part 620 is accommodated in the first bulging part 123. The first bulging part 123 is accommodated in the second bulging part 360, and the second bulging part 360 abuts the suction cover 400, so that the heating part 620 approaches the suction cover 400 as much as possible. When the heating part 620 generates heat, a heat transfer path of the heating part 620 can be shortened, so that the heat generated by the heating part 620 can be quickly conducted to the human breast through the first bulging part 123 and the suction cover 400, thereby increasing the heat conduction speed of the heating part 620, reducing loss during heat transfer, and improving a utilization rate of energy.

It should be understood that other undescribed parts of the breast pump in the fifth and sixth examples may be the same as the corresponding parts described with reference to the first to fourth examples, and will not be repeated here.

The above-described examples can be combined in any way. To simplify the description, not all possible combinations of the technical features in the above-described examples are described. However, as long as there is no contradiction in the combination of these technical features, they should be considered to be within the scope of this manual.

The above-described examples only express several implementation modes of the present disclosure, and their descriptions are relatively specific and detailed, but they should not be construed as limiting the scope of the patent disclosure. It should be noted that, for those of ordinary skill in the art, several modifications and improvements can be made without departing from the concept of the present disclosure, and these all fall within the protection scope of the present disclosure. Therefore, the protection scope of this patent disclosure should be determined by the appended claims.

What is claimed is:

1. A breast pump comprising:
a suction assembly comprising a suction cover that is in fluid communication with a storage bin; and
a main body coupled to the suction assembly,
wherein at least one of the suction cover and the main body comprises one or more heating elements electrically coupled to a power source in the main body.

2. The breast pump of claim 1, wherein the one or more heating elements are disposed on the suction cover or the main body.

3. The breast pump of claim 1, wherein the one or more heating elements are at least partially embedded in the suction cover or the main body.

4. The breast pump of claim 1, wherein the main body and the suction cover are disposed on the storage bin.

5. The breast pump of claim 1, wherein the main body is disposed between the suction cover and the storage bin.

6. The breast pump of claim 1, wherein the suction cover comprises the one or more heating elements electrically coupled to the power source in the main body.

7. The breast pump of claim 1, wherein the main body comprises the one or more heating elements electrically coupled to the power source.

8. The breast pump of claim 1, wherein a storage cavity is provided between the suction cover and the storage bin, and a liquid outlet hole is formed by at least one of the suction cover and the storage bin, and wherein the liquid outlet hole is in fluid communication with the storage cavity.

9. The breast pump of claim 8, wherein the suction cover comprises a fitting part and a mounting part, wherein the mounting part is protrudingly disposed on the fitting part and inserted into the storage bin, and wherein the fitting part abuts the storage cavity to seal the storage cavity.

10. The breast pump of claim 8, wherein the one or more heating elements comprise a heating part and a conductive part connected to each other, wherein the conductive part is electrically coupled to the main body.

11. The breast pump of claim 10, wherein the suction cover is provided with a liquid inlet channel, and projections of the conductive part, the liquid inlet channel and the liquid outlet hole are aligned on a same line.

12. The breast pump of claim 10, wherein the heating part is attached to an outer surface of the suction cover.

13. The breast pump of claim 10, wherein the conductive part comprises a pin with a column structure, and wherein, in a working state, the conductive part extends along a direction of gravity and abuts a corresponding pin on the main body, enabling the main body to supply power to the conductive part and the heating part.

14. The heating breast pump of claim 10, wherein the one or more heating elements further comprise a mounting base, wherein the mounting base is detachably connected to the main body, and the conductive part is connected to the heating part and extends through the mounting base.

15. The breast pump of claim 1, wherein the one or more heating elements comprise a temperature sensor electrically coupled to the main body.

16. The breast pump of claim 1, wherein the one or more heating elements are embedded in the suction cover or the main body by in-mold injection molding.

17. The breast pump of claim 1, wherein the one or more heating elements comprise a spiral resistance wire.

18. The breast pump of claim 17, wherein the one or more heating elements comprise a heat conducting sheet that adheres to a flexible heating sheet.

19. The breast pump of claim 1, wherein the one or more heating elements are electrically coupled with the main body.

20. The breast pump of claim 1, wherein the one or more heating elements are embedded in the suction cover, an adjustment plug is inserted into the suction cover and covers a heating part of the one or more heating elements, and wherein the adjustment plug is provided with an annular step surface, and the heating part abuts the annular step surface.
